(19) United States
(12) Patent Application Publication
(10) Pub. No.: US 2002/0026132 A1
(43) Pub. Date: Feb. 28, 2002

(54) Device for spinal column treatment

(76) Inventor: Norbert Schipke, Maxhutte-Haidhof (DE)

Correspondence Address: BIERMAN MUSERLIAN AND LUCAS, 600 THIRD AVENUE, NEW YORK, NY, 10016

(21) Appl. No.: 09/768,750

(22) Filed: Jan. 24, 2001

(30) Foreign Application Priority Data
Jul. 10, 1998 (DE) 19830934.1

(51) Int. Cl. A61F 5/00; A61H 1/00

(52) U.S. Cl. 601/90; 606/240; 601/80

(57) ABSTRACT

The invention relates to a novel device for spinal column treatment in the form of a pallet and is characterized by a lying surface spring mounted on a stand or frame with at least one oscillation generator to generate a three-dimensional oscillation of the lying surface in all three mutually perpendicular spatial axes.

The invention relates to a device for spinal column treatment in the form of a pallet having a lying surface spring mounted on a stand or frame, having at least one oscillation generator to generate a three-dimensional oscillation of the lying surface in all three mutually perpendicular spatial axes, and having a vertically adjustable foot rest for stepped support of the patient.

There is a known device of this kind (brochure “TheraGe Schmerztherapie ohne Medikamente . . . ” [TheraGe pain therapy without drugs . . . ]). In the known device, the lying surface, moved by the oscillation generator, is provided detached from the adjustable foot rest, i.e. the foot rest does not move together with the lying surface.

The object of the invention is to disclose a device affording a still further improved therapeutic effect. To accomplish this object, a device is configured according to claim 1.

In the device according to the invention, the foot rest participates in the vibratory motions of the lying surface. Among other things, this achieves a substantially improved mobilization of the extremities, since arms and legs are directly exposed to the oscillations generated by the oscillation generator. Thus, using the device according to the invention, surprisingly enough, a correction of the iliosacral joints is possible without manual intervention, by vibratory treatment alone. Furthermore, the configuration according to the invention serves better for lymphatic decongestion than was possible with the known device, since according to the invention the upper and lower extremities of a patient's body are acted upon alike by the vibratory oscillation.

The device according to the invention is distinguished further by an especially compact construction, simple operation, and in particular an optimal therapeutic effect.

Refinements of the invention are the subject of the subsidiary claims. The invention will be illustrated below in more detail in connection with the figures, with an embodiment by way of example. In the drawing,

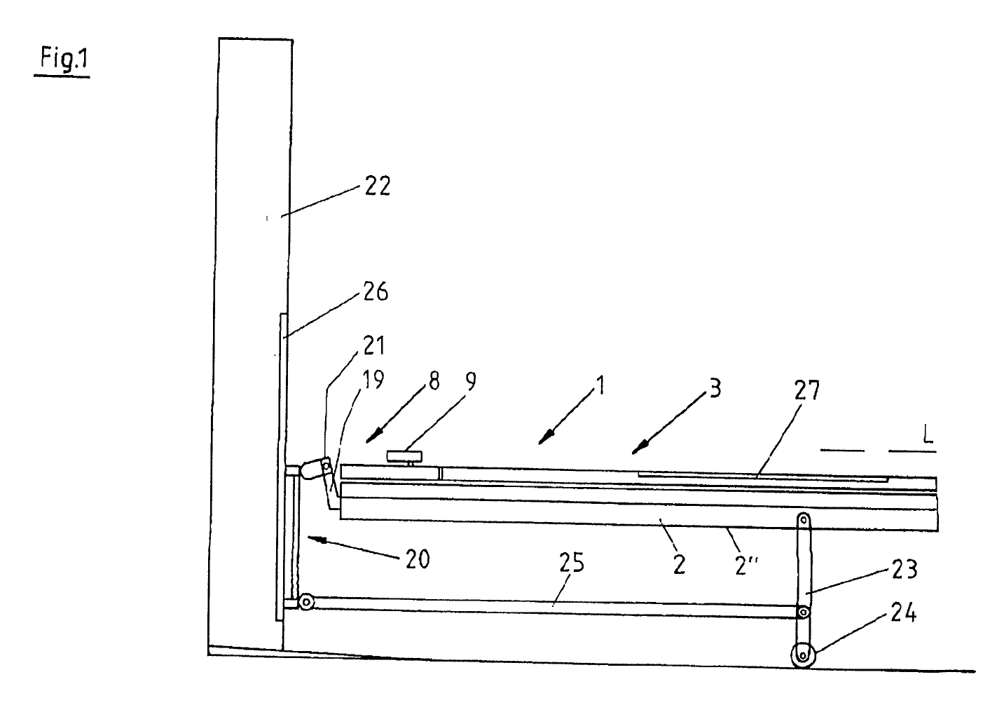

FIG. 1 shows a device according to the invention in side view;

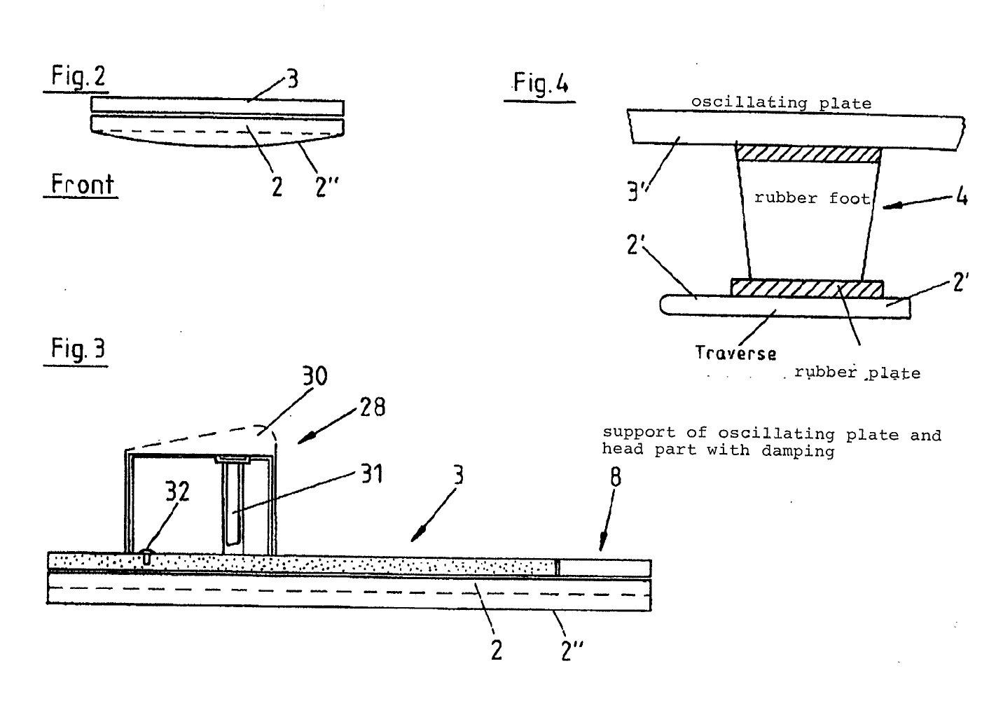

FIG. 2 shows a rear view of the device of FIG. 1;

FIG. 3 shows a partial view of the device from the side, together with a foot part;

FIG. 4 shows an enlarged representation of the mounting of the lying surface;

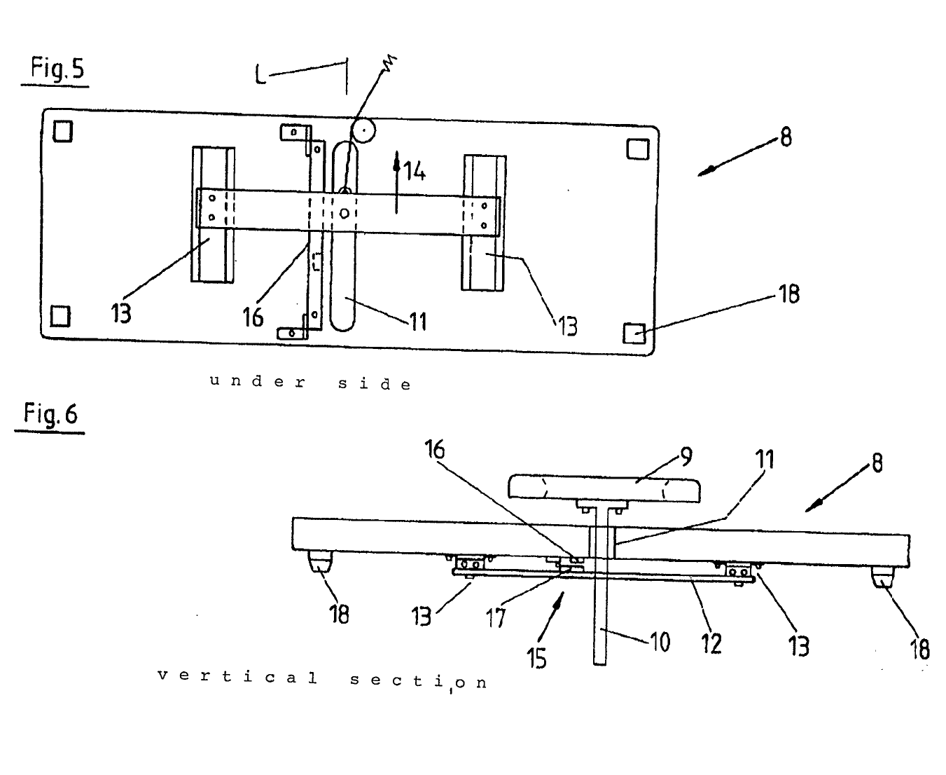

FIGS. 5 and 6 show the head part of the pallet together with a head rest, in side view and in bottom view;

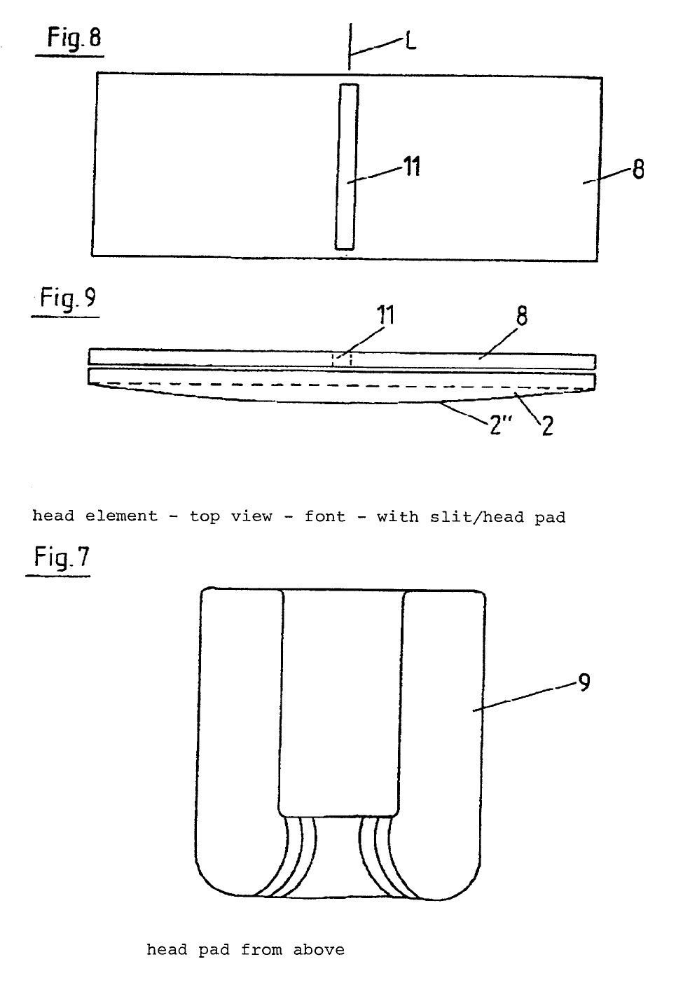

FIG. 7 shows an enlarged partial representation of the head rest in top view;

FIG. 8 shows a top view of the head rest of FIGS. 5 and 6;

FIG. 9 shows a side view of the head part or element;

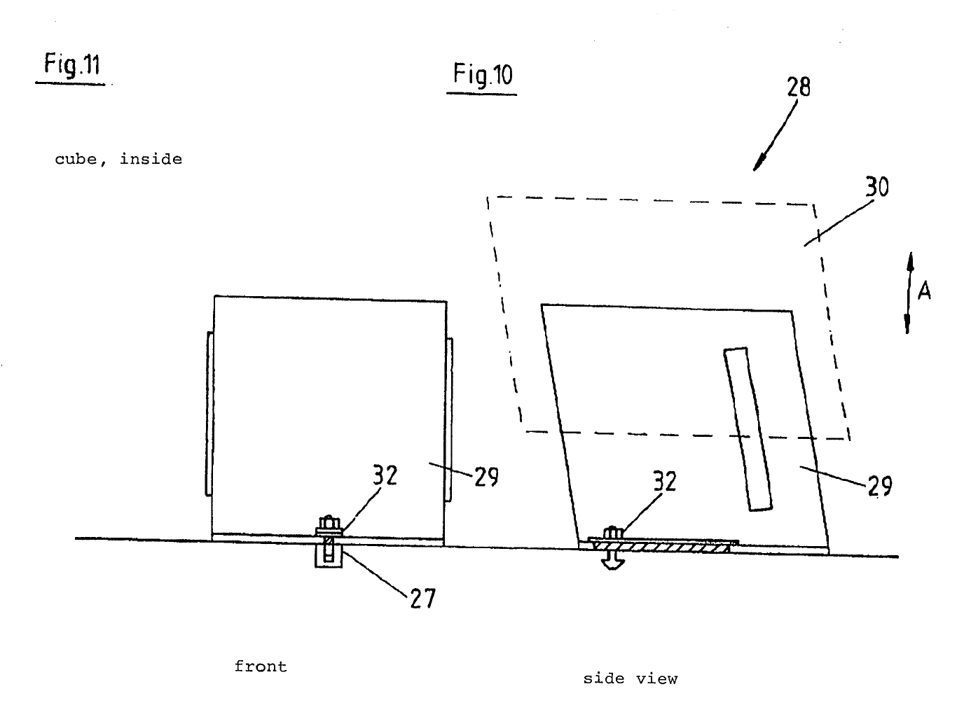

FIGS. 10 and 11 show the guide piece of the foot part in side view and in a view rotated 90°;

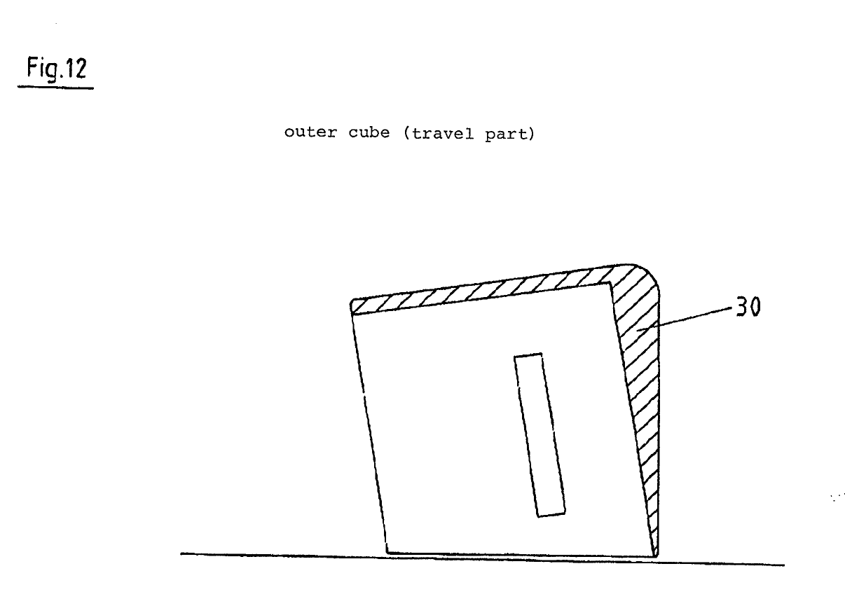

FIG. 12 shows the upper, vertically adjustable member of the foot part;

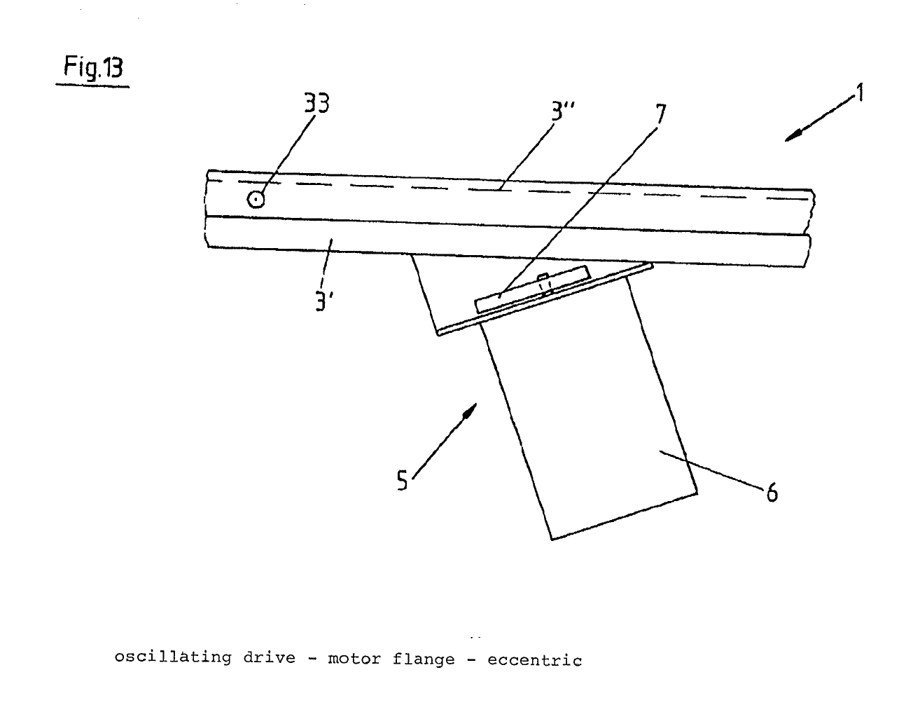

FIG. 13 shows an oscillation drive with eccentric provided on the under side of the pallet.

The device for spinal column treatment as represented in the figures is constructed as a pallet 1, and consists essentially of a frame or stand 2 on the top of which a lying surface 3 for the shoulder, torso and leg regions of the patient to be treated is provided, and elastically or spring mounted in all three spatial axes, with the aid of rubber bumpers or feet 4, one of which is shown in detail in FIG. 4. The rubber feet 4 are each attached firstly to the stand or housing 2 or to a frame 2′ thereon, and secondly, above the frame 2′, to the lying surface 3 or to an oscillating plate 3′ forming the substrate of the lying surface. The oscillating plate 3′ is upholstered on top at 3″. On the under side of the vibrating plate 3′, the vibratory drive 5 is attached as well, consisting of an electric motor 6 and a mass 7 eccentrically mounted on the motor shaft, and revolving when the motor 6 is switched on about the motor shaft making an angle with the plane of the oscillating plate 3′, thus setting the plate 3′ in an oscillatory motion having components in all three spatial axes, namely the two axes determining the plane of the oscillating plate 3′ and the spatial axis perpendicular thereto.

On the frame 2′ [of the] housing 2, at the head end of the pallet 1, a head element 8 is also provided, preferably removable. Between the lying surface 3 and the head element 8, there is a gap, so that vibratory motions of the lying surface 3 are not transmitted to the head element 8.

On the head element 8, a head rest 9, upholstered and serving to support the patient's head, is provided, displaceable in lengthwise direction L of the pallet 1. The head rest 9 is fixed for this purpose to the upper end of a supporting rod 10 extending through the slit 11 of the head element 8 on the under side of the he-ad element, extending in the direction of the lengthwise axis L, and there attached, preferably vertically adjustable, to a flat leaf-spring-like supporting member 12, whose lengthwise extent is perpendicular to the lengthwise axis L and fixed at each end to a ball guide 13, likewise extending in the direction of the lengthwise axis L and providing guidance in that direction.

By the force of a spring or a weight which (force) is indicated in FIG. 5 by an arrow 14, the head rest 9 is prestressed away from the lying surface 3 in lengthwise direction L, so that to achieve the desired therapeutic effect on the person lying on his back on the pallet 3, with his head on the head rest 9, when the lying surface 3 is oscillated (by the drive 5) some tension is exerted at the same time on the patient's head.

To avoid an undesired displacement or moving away of the head rest 9 by the force 14 of the weight or spring when the head is raised briefly, i.e. to compensate the action of the force 14 when the head rest 9 is not under load, in the embodiment shown a brake or locking device 15 is provided, becoming active when the head rest 9 is relieved, i.e. locking the head rest 9 in that position which the head rest will assume just when the load is removed. The brake or locking device 15 in the embodiment shown consists essentially of a rail 16 extending at the under side of the head element 8 parallel to the slit 11 on one side of the slit, to wit between the- under side of the head element 8 and the supporting member 12.

On the side towards the rail 14, the supporting member 12 is provided with a brake block 17, which when the head rest 9 is not under load will lie pressed against the rail 16 and thereby block the head rest 9 from the action of the force 14 of the spring or weight. When the head rest 9 is loaded, the supporting member 12 yields somewhat in the middle, bringing the brake block 12 out of engagement with the rail 16 and thereby allowing the force 14 of the spring or weight to act fully on the head rest 9; in installed condition, the head element 8 is held to the frame 2′ of the housing 2 by four elastic feet 18.

As the figures show, the pallet 1 is of very flat construction; in order to accommodate functional elements of considerable size in the housing 2, namely the oscillatory drive 5 and the supporting rod 10 of the head rest 9, the under side of the housing 2 is convexly curved at 2″, about an imaginary axis parallel to the lengthwise axis L.

Another peculiarity of the pallet 1 consists in that the housing or frame 2 is rendered swingable at the end comprising the head element 8 on a bearing 20 with the aid of a support 19, namely using joint pins 21 about a horizontal axis perpendicular to the longitudinal axis L. The bearing 20 is mounted on a building wall 22.

At the end away from the bearing 20, on both lengthwise sides of the frame 2, a foot 23 is provided, swingable by its upper end about a horizontal axis perpendicular to the lengthwise extent L, having a roller 24 at its lower end. Between the feet 23 and the bearing 20, a rod 25 is provided, swingably attached at one end to the bearing 20 and at the other end to the feet 23, in each instance about a horizontal axis extending perpendicular to the lengthwise extent L.

In the embodiment shown, the bearing 20 is attached to the housing wall 22 vertically adjustable with the aid of a guide rail 26, so that by changing the vertical position of the bearing 20, a differential obliquity of the pallet 1 is made possible. By means of the strut or rod 25, the feet 23 can be arranged vertical or at least approximately vertical in any oblique position of the pallet.

At the end of the lying surface 3 away from the head part 8, the same is provided with a holding or adjusting rail 27 accessible at the top of the lying surface 3 and extending in lengthwise direction L, to which rail a cubical foot part 28 can be attached, adjustable in the direction of the lengthwise axis L, for a stepped support of a patient. The foot part 28 consists of an inner member 29 and an outer member 30 vertically adjustable on the inner member 29. In the embodiment shown, the inner member 29 and the outer member 30 each has an outer and inner cross-section, respectively, that is, substantially square. The outer member 30 forms the support for the patient's feet and is accordingly constructed as a padded cushion on the outside. A preferably electrical motor drive 31 makes possible a motorized vertical adjustment of the outer part 30 relative to the inner part 29 and hence to the lying surface 3.

To supply and control the drive 31, electric contacts are accommodated in the guide rail 23, to which the electrical connection is made by way of the holding or anchorage elements 32 specially constructed for the purpose, mechanically anchoring the under part 29 in the guide rail 27. The adjusting drive in the embodiment shown takes the form of a low-voltage (e.g. 24 volts) drive. The lying surface 3 or its padding 3″ is preferably heatable, namely by means of an electric heater 33 installed in the padding 3″.

Instead of the drive 5 described above, other drives may be provided, for example such as comprise several motors 6, each having an eccentric mass 7, in which case the motors 6 are arranged with the axes of their shafts in different spatial axes, namely for example in three mutually perpendicular spatial axes, of which e.g. two define the plane of the oscillating plate 3 and one lies perpendicular to that plane. The use of three independent motors or drives 6 also makes it possible to generate the different patterns of vibratory motion by differential triggering of these drives, in order for example to achieve an optimum motion of the lying surface 3 for the particular application.

The invention has been described above in terms of an embodiment by way of example. It will be understood that many changes or modifications are possible without thereby departing from the inventive idea underlying the invention.

What is claimed is:

1. Device for spinal column treatment in the form of a pallet (1) having a lying surface (3) spring mounted on a stand or frame (2), having at least one oscillation generator (5) to generate a three-dimensional oscillation of the lying surface in all three mutually perpendicular spatial axes, and having a vertically adjustable foot rest (28) for stepped support of the patient, characterized in that the foot rest attached to the lying surface shares the vibratory motion of the lying surface and is adjustable in lengthwise direction of the lying surface.

2. Device according to claim 1, characterized in that for attachment of the foot rest (28) to the lying surface, at least one fastening rail (27) extending in lengthwise direction (L) is provided.

3. Device according to claim 2, characterized in that contacts are provided in the guide rail (27) for an electric drive (31) of the vertical adjustment of the foot rest (28).

4. Device according to claim 1, characterized in that a head element (8) having a head rest (9) is provided on the stand, mounted displaceable in lengthwise direction (L) of the device (1) and capable of being acted upon by a force (14), and in that means are provided to arrest the head rest (9) when the oscillation drive is switched off and/or when the head rest (9) is relieved.

5. Device according to claim 4, characterized in that the means of arresting the head rest (9) automatically arrest in their position upon release of the head rest.

6. Device according to claim 1, characterized in that the lying surface (3) is heatable.

7. Device according to claim 1, characterized in that the stand (2) of the pallet (1) is articulated at one end to a vertically adjustable bearing (20).

8. Device according to claim 1, characterized in that for the lying surface (3), at least two independently controllable drives (2) are provided.
